(19) United States
(12) Patent Application Publication
(10) Pub. No.: US 2025/0090627 A1
(43) Pub. Date: Mar. 20, 2025

(54) NUTRITIONAL POLYPEPTIDE HAVING BRANCHED-STRUCTURE WITH EFFICIENT BROAD-SPECTRUM ANTIBACTERIAL AND ANTIFUNGAL FUNCTIONS

(71) Applicants: ZHEJIANG UNIVERSITY, Hangzhou (CN); SHAOXING RESEARCH INSTITUTE OF ZHEJIANG UNIVERSITY, Shaoxing (CN)

(72) Inventors: Changyou GAO, Hangzhou (CN); Wangbei CAO, Hangzhou (CN); Xiaofei DONG, Hangzhou (CN)

(21) Appl. No.: 18/963,764

(22) Filed: Nov. 29, 2024

Related U.S. Application Data
(63) Continuation of application No. PCT/CN2022/097173, filed on Jun. 6, 2022.

(30) Foreign Application Priority Data
May 30, 2022 (CN) 202210600563.9

(51) Int. Cl. A61K 38/16; A61K 9/06; A61K 47/32; A61K 47/34; A61K 47/42; A61P 31/04; A61P 31/10

(52) U.S. Cl. CPC A61K 38/16; A61K 9/06; A61K 47/32; A61K 47/34; A61K 47/42; A61P 31/04; A61P 31/10

(57) ABSTRACT

A nutritional polypeptide having branched-structure with efficient broad-spectrum antibacterial and antifungal functions provided. The nutritional polypeptide having branched-structure is a polypeptide containing L-lysine or D-lysine moieties having a molecular weight of 3000-7000 Da. The branched-structured polypeptide has a highly efficient broad-spectrum antibacterial effect against bacteria, fungi and mycoplasma at low concentrations without developing resistance. Compared with conventional antimicrobials, the disclosed polypeptides have excellent biocompatibility, are non-cytotoxic, have no adverse effects on animals by intravenous injection, and can satisfy the requirements of intravenous antimicrobials in vivo. The degradation product is lysine, a nutrient essential to the organism, which has no toxic side effects. The branched-structured polypeptide is chemically synthesized throughout without the need of biofermentation processes, and is physically, chemically and biologically stable. It can be physically or chemically combined with other materials, synthesized or processed into various forms of materials and retain efficient antimicrobial properties.

### CROSS-REFERENCE TO RELATED APPLICATIONS

The present application is a National Stage of International Application No. PCT/CN2022/097173 filed Jun. 6, 2022, which claims a priority to Chinese Patent Application No. 2022106005639, filed on May 30, 2022, both of which are hereby incorporated by reference in their entireties.

### TECHNICAL FIELD

The present disclosure belongs to the field of medical polymeric materials and relates to a nutritional polypeptide having branched-structure with efficient broad-spectrum antibacterial and antifungal functions.

### BACKGROUND

Microbial infections, particularly bacterial, fungal and mycoplasma infections, have been a significant problem that hinders human survival. In the early 20th century, bacterial epidemics were a significant cause of death worldwide. In contrast, fungal and mycoplasma infections have hardly been considered. With the development of public health and biomedical technology, penicillin and many other antibiotic drugs are developed, and many bacterial infection problems are effectively resolved. But, at the same time, a dramatic rise in the rate of fungal infections is observed, which remains a significant threat to global human health. The number of drugs currently available to combat invasive fungal infections is very limited. There are only four types of drug molecules, including fluoropyrimidines and derivatives, polyenes, azoles, and echinocandins, that can treat systemic fungal infections in the clinic with the ability of targeting three different metabolic pathways. Other drugs such as the morpholine and allylamine based molecules are currently only used topically due to poor efficacy, or to severe potential adverse effects when administered systemically. Infections by mycoplasma should not be underestimated either. Unlike bacteria, mycoplasma does not have cell membranes, therefore antibiotics couldn't win the combat against it by attacking cell membranes. Mycoplasma infection is currently commonly treated with erythromycin and tetracycline.

Due to the use of the above antibacterial/fungal/mycoplasma drugs in the long term, the drug abuse causes the emergence of drug resistance, even multi-drug-resistant microorganisms. Even though antibiotics continue to be approved for use, drug-resistant microorganisms continue to emerge correspondingly, such as methicillin-resistant Staphylococcus aureus, resulting in a dramatic decrease in the broad-spectrum antibacterial capacity of existing antibacterial/fungal drugs. Currently the mycoplasma resistance to erythromycin and tetracyclin is up to 90%. In addition, conventional antibacterial molecules also suffer from other problems, such as water solubility, allergy, overdose, cytotoxicity, etc. Superior antimicrobials, such as 75% ethanol, hypochlorous acid, iodophors, etc., can only be used in the sterilization and disinfection of environment, medical devices, and biological surfaces, and are difficult to use for infections in vivo. Therefore, it is an ongoing object to obtain new antimicrobial agents with low or even non-toxic toxicity, a broad spectrum and high efficacy.

Currently, antibacterial agents available for organisms are typically divided into inorganic antibacterial agents, organic antibacterial agents, and natural antibacterial agents. Inorganic antimicrobials include, but are not limited to, silver, copper, zinc, gold, and compounds thereof, which are used in ionic or nanoparticulate form. These antibacterial agents can achieve strong antibacterial effects by destroying film layers, photothermal, photodynamic, etc., but face major problems in that they are difficult to degrade or metabolize in the body, are easily enriched in the body and cause chronic toxicity over a long period of time. Inorganic nanoparticles have been reported to adsorb nonspecifically to serum albumin in blood in vivo, and silver nanoparticles have also been reported to cause damage to intracellular genes, especially in vigorously dividing cells. Organic antibacterial agents include synthetic low-molecular and high-molecular organic antibacterial materials. Low molecular species are mainly quaternary ammonium salts and phenolic alcohol esters, and the polymers are mainly polymers containing antibacterial groups or structures, such as cationic polymers, quaternary ammonium salt polymers, which have advantages of various types, designable structure, wide application range and large-scale production, but have disadvantages that the carbon-carbon chain of the polymer cannot be degraded, and the composition and structure of the product after degradation are complex, resulting in difficulty in predicting toxicity to organisms. Natural antibacterial agents mainly include plant-derived essential oils, chitosan, catechol, and the like. Unlike synthetic antibacterial agents, natural antibacterial agents are obtained by direct extraction and purification by humans from nature, have a long history of discovery and use, and have good biocompatibility, but have disadvantages of poor stability, low yield, narrow antibacterial spectrum, low antibacterial efficiency, and the like. The development of an antimicrobial agent that combines the advantages described above is therefore a difficult and hot-spot problem in the current research of antimicrobial substances.

Antimicrobial peptides, also known as polypeptide antibiotics, refer to a class of short peptides with an amino acid number of less than 100 and broad-spectrum antibacterial properties. Initially in the 1980s, Boman, a Swedish scientist, isolated the first antibacterial polypeptide, known as cecropin, from the pupal lymphatic fluid of Cecrombi. Since then, humans have discovered thousands of antimicrobial peptides in almost all organisms, which is an important defense of organisms against invasion by foreign pathogens.

Antimicrobial peptides have a number of advantages over traditional antimicrobial drug molecules and are therefore considered to have great promise for antimicrobial applications. Firstly, antimicrobial peptides have a highly effective, broad-spectrum antimicrobial ability to kill objects including Gram-positive bacteria, Gram-negative bacteria, fungi, parasites, mycoplasma, and the like, and are almost non-resistant. And a few of them have the effect of killing viruses and tumor cells. Secondly, most antimicrobial peptides have no or low toxicity to cells within the organism in the concentration range at which they exert an antimicrobial effect. Thirdly, compared to macromolecular bioantimicrobial formulations such as immunoglobulins, they have small molecular mass, good water solubility, faster diffusion rates in the organism, and better thermal stability. At the same time, the biosynthesis rate thereof is much greater than that of immunoglobulins. Therefore, antimicrobial peptides, also known as “natural antibiotics,” are expected to overcome the growing problem of antibiotic resistance.

Compared to traditional antibiotics, antimicrobial peptides have different pharmacology. Traditional antibiotics achieve sterilization by inhibiting or interfering with the vital activities or biological functions necessary for microbial metabolism, such as obstructing the synthesis of bacterial essential protein, the synthesis of bacterial cell wall or inhibiting the activity of bacterial intracellular enzymes. However, the bacteria can generate an ability to cope with the antibiotic and pass it on from generation to generation by mutating at least one of the relevant genes. Antimicrobial peptides act primarily on bacterial cell membranes, resulting in increased membrane permeability, thereby killing bacteria or other types of pathogens. However, it is difficult for bacteria to change the structure of the bacterial cell membrane by changing only one gene in the above manner, and therefore, the antimicrobial peptide can be considered as not producing resistance while killing pathogenic microorganisms. Secondly, antimicrobial peptides are selective for their target of action, having an antimicrobial effect only on bacteria, fungi and mycoplasma, and having no effect on normal eukaryotic cells. The reason is that bacteria/fungi/mycoplasma and human cells have different cell membrane structures, the human cell membrane contains large amounts of cholesterol, and the presence of cholesterol makes the membrane structure more stable. In addition, higher animals present a highly developed cytoskeletal system that is also resistant to the effects of antimicrobial peptides. Currently, antimicrobial peptides have been used in certain practices and good results in the fields of agriculture, medicine, food and cosmetics.

In the field of agriculture, the excellent antibacterial effect of antimicrobial peptides makes it promising to replace the use of antibiotics in feed, avoiding microbial resistance caused by abuse of antibiotics, while its degradability avoids the accumulation of antibiotics in animals, reducing the impact on agricultural product quality and consumer health. In the field of foods and cosmetics, antimicrobial peptide bacteriocins, e.g. isolated from lactic acid bacteria, can inhibit the growth of various pathogenic microorganisms in foods and cosmetics and act as preservatives.

However, there are still challenges in the large-scale production of antimicrobial peptides (AMPs). Due to their low molecular weight, the extraction and purification of natural AMPs directly from animal or plant tissues present certain difficulties. As a result, chemical synthesis and genetic engineering fermentation methods have become the primary approaches to obtain AMPs. However, chemical synthesis requires maintaining consistent peptide sequences, which leads to high cost in large-scale production. In contrast, genetic engineering involves expressing AMP genes directly in microorganisms. This approach faces limitations due to the susceptibility of AMPs to protease degradation and the potential toxicity of the expressed product to the host bacteria, which hampers high-level gene expression. Thus, improving production efficiency and reducing costs are pressing issues that need to be addressed for the large-scale application of AMPs. On the other hand, compared to traditional antibiotics, the antibacterial activity of some AMPs has not yet reached an ideal performance. Modifying existing AMP sequences or designing new antimicrobial peptides with novel polymeric amino acid is an effective strategy to enhance their activity.

### SUMMARY

It is an object of the present disclosure to disclose a nutritional polypeptide having branched-structure with efficient broad-spectrum antibacterial and antifungal functions. Firstly, the branched-structured polypeptide is composed entirely of lysine, without considering the control of the amino acid sequence structure during synthesis, and thus is easy to prepare at a low cost. Secondly, the branched-structured polypeptides have good biocompatibility, including good cytocompatibility, histocompatibility, and hemocompatibility, compared to other chemically synthesized guanidine, quaternary ammonium polymer antibacterial agents. It has highly effective antibacterial properties at a low concentrations, achieving an antibacterial rate of 90% or more at a concentration of 0.001% (10 μg/mL), and can be used in vivo without toxicity to normal tissues, blood and cells. Compared to medical alcohol, hydrogen peroxide, quaternary ammonium antibacterial agents, and other antimicrobial peptides, the branched-structured polypeptide has a wider range of antimicrobial applications, including antimicrobial in vitro, skin wound surface antimicrobial, and antimicrobial injection in vivo. Its degradation product is lysine, one of the natural amino acids, which is an amino acid essential to cells or biological tissues after performing an antibacterial function. The branched-structured polypeptides can be physically or chemically combined with other materials, to synthesize and process into the form of antimicrobial hydrogels, antimicrobial polymers, antimicrobial metal devices, antimicrobial ceramics, antimicrobial nanoparticles, antimicrobial microgels, and the like, and have a wide range of applicable scenarios and application forms as a biomedical antimicrobial material.

The present disclosure provides a nutritional polypeptide having branched-structure with efficient broad-spectrum antibacterial and antifungal function, which is a polypeptide containing L-lysine or D-lysine moieties, and can be polymerized from L-lysine, D-lysine or both amino acids or hydrochlorides thereof as starting materials.

The polypeptide according to the disclosure has a number average molecular weight in the range of 3000-7000 Da, with a polydispersity index typically less than 1.2;

The polypeptide according to the disclosure has superior antibacterial ability against bacteria, fungi and mycoplasma in an aqueous solution environment.

The bacteria involved includes Gram-positive bacteria exemplified by Staphylococcus aureus and methicillin-resistant Staphylococcus aureus and drug-resistant variant strains thereof, Gram-negative bacteria exemplified by Escherichia coli and Pseudomonas aeruginosa and drug-resistant variant strains thereof.

The fungi involved includes yeast fungi exemplified by Saccharomyces and Candida albicans and drug-resistant variant strains thereof, mould fungi exemplified by Aspergillus niger and Aspergillus flavus and drug-resistant variant strains thereof.

The Mycoplasma involved includes Mycoplasma vulgaris as well as Mycoplasma resistant varieties.

The aqueous environment refers to all water-solvent environments including, but not limited to, pure water, deionized water, phosphate buffer solution, physiological saline, cell culture medium, blood, interstitial fluid, and the like.

In the antimicrobial applications contemplated, the concentration of the polypeptide ranges from 10 μg/mL to 20 μmg/mL to ensure its antimicrobial and biocompatible effects. Within this concentration range, the polypeptide can be dissolved in a cell culture medium and co-cultured with the cells; or the polypeptide is dissolved in physiological saline and injected into the animal.

The polypeptide according to the present disclosure is a nutritional antibacterial polypeptide, which is degraded into lysine, a nutrient, which is necessary for a living body, and participates in normal physiological activities of a living body after exerting an antibacterial effect.

The polypeptide of the present disclosure does not require biological fermentation and is chemically synthesized throughout, with a stable structure that is not easily denatured, and can be combined with other materials by various physical or chemical means to form an antibacterial complex.

Further, the physical mean may be physical entrapment, electrostatic adsorption, non-specific adsorption, co-extrusion, or the like.

Further, the chemical mean may be: (1) chemical graft modification with polymer, metal and ceramic bulk materials; (2) acting as a crosslinker between polymer chains, and forming a polymer network; and (3) various binding means, such as forming a covalent bond with other small molecules, by using amino groups of the polypeptide.

Further, the complex may be in the form of a hydrogel, a polymer, a metal, a nanoparticle, a microgel, or the like.

The present disclosure has the following advantages:

The present disclosure discloses a branched-structured polypeptide and the use thereof in antibacterial. The polypeptide has highly effective antibacterial properties at a low concentration, i.e. it has significant antibacterial function against Gram-positive bacteria, Gram-negative bacteria, yeast fungi, mould fungi, mycoplasma, and corresponding resistant varieties thereof, at 0.001% concentration (10 μg/mL), thus can be developed into a variety of products with excellent antibacterial function. Meanwhile, the polypeptide has excellent biocompatibility and blood compatibility in vivo, and the degradation product thereof is one of the essential amino acids of organisms. It has no toxic effect on organisms, and solves the problems of drug resistance, high price and poor biocompatibility of the traditional antibacterial agents. In addition, the antimicrobial peptide having a branched structure disclosed in the present disclosure can be dissolved in an injection solution and injected into the living body by intravenous injection or intramuscular injection, for antibacterial treatment of blood infection such as sepsis or septicemia in vivo.

### BRIEF DESCRIPTION OF DRAWINGS

FIG. 1A shows the antibacterial effect of the branched-structured polypeptide of the present disclosure (prepared by Method 1) against Staphylococcus aureus, Escherichia coli, Pseudomonas aeruginosa and Candida albicans at a concentration of 10⁸ CFU/mL (the concentrations of the polypeptide are 250, 500, 750 and 1000 μg/mL, respectively).

FIG. 1B shows the antibacterial effect of the branched-structured polypeptide of the present disclosure (prepared by Method 2) against Staphylococcus aureus, Escherichia coli, Pseudomonas aeruginosa and Candida albicans.

FIG. 2 shows the antibacterial effect of the branched-structured polypeptides of the present disclosure against methicillin-resistant Staphylococcus aureus at a concentration of 10⁸ CFU/mL.

FIG. 3 shows the antibacterial effect of the branched-structured polypeptides of the present disclosure against methicillin-resistant Staphylococcus aureus at a concentration of 10⁹ CFU/mL.

FIG. 4 shows the results of the cell viability of the branched-structured polypeptides of the present disclosure after culture of fibroblast cells.

FIG. 5 shows the cell concentrations of white blood cells, neutrophils, monocytes and lymphocytes in rat blood 24 h after injection of the branched-structured polypeptide of the present disclosure at 1 mg/ml.

FIG. 6 shows the release curve of the branched-structured polypeptides within one week.

### DETAILED DESCRIPTION OF EMBODIMENTS

The present disclosure will now be described in detail below reference to the examples. The examples are given only for the purpose of illustrating the present disclosure and are not to be construed as limiting the scope of the present disclosure, and insubstantial modifications and variations thereof will occur to those skilled in the art in light of the above teachings. In case of no conflict, the embodiments in the present disclosure and the features in the embodiments may be combined with each other.

### Example 1

A nutritional polypeptide having branched-structure with efficient broad-spectrum antibacterial and antifungal functions can be prepared in the following two ways.
Method 1:2000 g of L-lysine sulfate, 700 g of solid potassium hydroxide and 2 g of tripyridine boronic acid were added into a 5 L reaction kettle equipped with a stirrer, an internal thermometer, a gas inlet tube, a condenser and a collector; the mixture was gradually heated to an internal temperature of 140° C. under stirring and nitrogen protection, and the heating was stopped after 20 hours; a hyperbranched polylysine crude product was discharged from the bottom collector while hot. The crude product was added to 1500 mL of ethanol to obtain a suspension containing the hyperbranched polylysine after thorough mixing. The suspension was centrifuged at 3000 rpm to obtain an ethanol solution of the hyperbranched polylysine, and the solution was injected into an aerosol drying tower and dried to obtain pure hyperbranched polylysine powder.

The number average molecular weight and PDI of the hyperbranched polylysine prepared in this example were 5299 Da and 1.11, respectively, as measured by GPC.
Method 2:2000 g of D-lysine sulfate, 700 g of solid potassium hydroxide and 2 g of tripyridine boronic acid were added into a 5 L reaction kettle equipped with a stirrer, an internal thermometer, a gas inlet tube, a condenser and a collector; the mixture was gradually heated to an internal temperature of 140° C. under stirring and nitrogen protection, and the heating was stopped after 22 hours; a hyperbranched polylysine crude product was discharged from the bottom collector while hot. The crude product was added to 1500 mL of ethanol to obtain a suspension containing the hyperbranched polylysine after thorough mixing. The suspension was centrifuged at 3000 rpm to obtain an ethanol solution of hyperbranched polylysine, and the solution was injected into an aerosol drying tower and dried to obtain pure hyperbranched polylysine powder.

The number average molecular weight of the hyperbranched polylysine prepared in this example was 5880 Da.

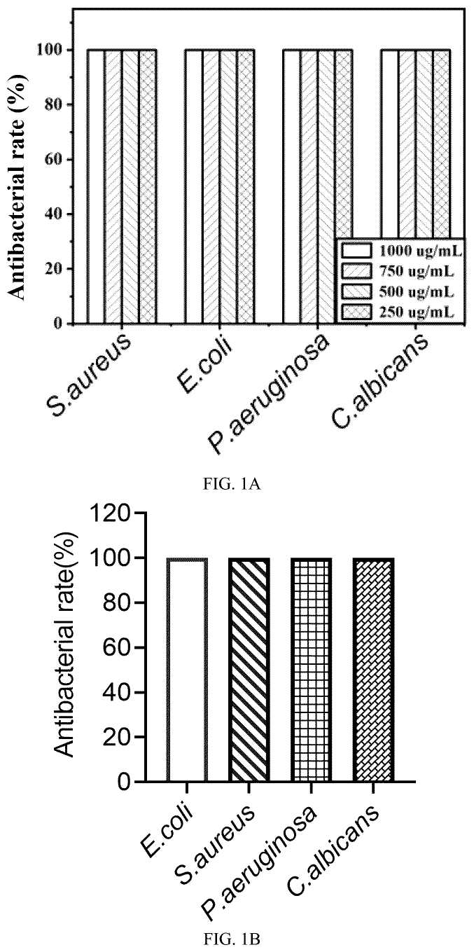

The antibacterial effect thereof against Gram-positive bacteria, Gram-negative bacteria and fungi is demonstrated as follows:
the polypeptides produced by the above method 1 and method 2 were added at a final polypeptide concentration of 250 μg/mL in Staphylococcus aureus, Escherichia coli, Pseudomonas aeruginosa and Candida albicans at a concentration of 10⁸ CFU/mL, respectively, and co-cultured at 37° C. for 24 hours. The results are as shown in FIG. 1A and FIG. 1B. The resulting polypeptides had an antibacterial effect of 100% on the above bacteria and fungi, and the polypeptides produced by the present disclosure were effective in killing bacteria and fungi.

### Example 2

A Nutritional Polypeptide Having Branched-Structure with Efficient Broad-Spectrum Antibacterial and Antifungal Functions has an Antibacterial Effect Against Drug-Resistant Bacteria as Follows:

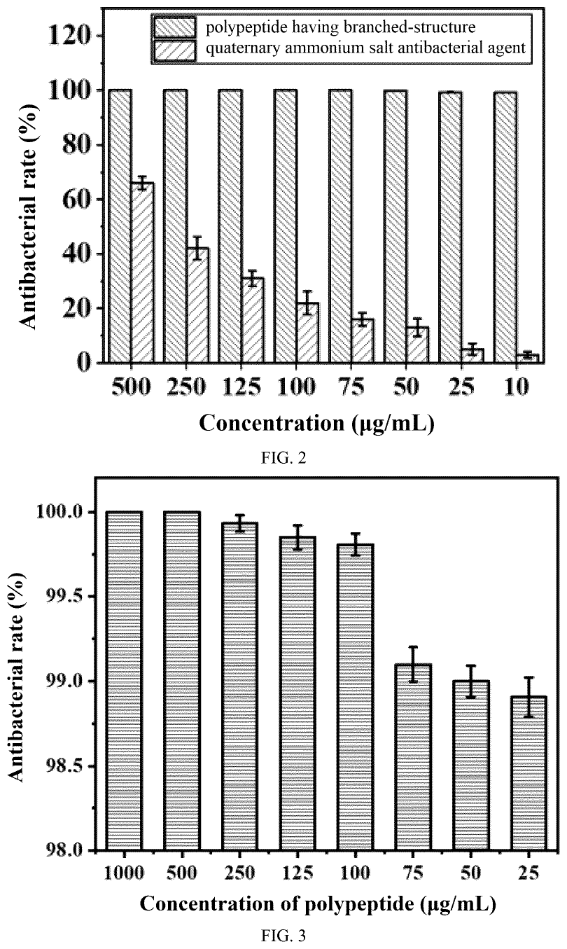

The above polypeptide (prepared by Method 1) was added in methicillin-resistant Staphylococcus aureus solutions at concentrations of 10⁸ and 10⁹ CFU/mL respectively according to the polypeptide concentration gradient, and then co-cultured at 37° C. for 24 hours. The results are as shown in FIG. 2 and FIG. 3. The polypeptide was effective in killing methicillin-resistant Staphylococcus aureus in the effective concentration range. The antimicrobial rate of 100 μg/mL of polypeptide solution could reach up to 100% (FIG. 2) and 98% or more (FIG. 3) respectively. Under the same conditions, the quaternized antimicrobial agent at a corresponding concentration was far less effective than the branched-structured polypeptide when facing a concentration of 10⁸ CFU/mL, whereas penicillin G at a concentration of 100 μg/mL showed only an antimicrobial rate of less than 10% in both sets of antimicrobial experiments.

### Example 3

A Nutritional Polypeptide Having Branched-Structure with Efficient Broad-Spectrum Antibacterial and Antifungal Functions has the Safety (Cytotoxic) Properties as Follows:

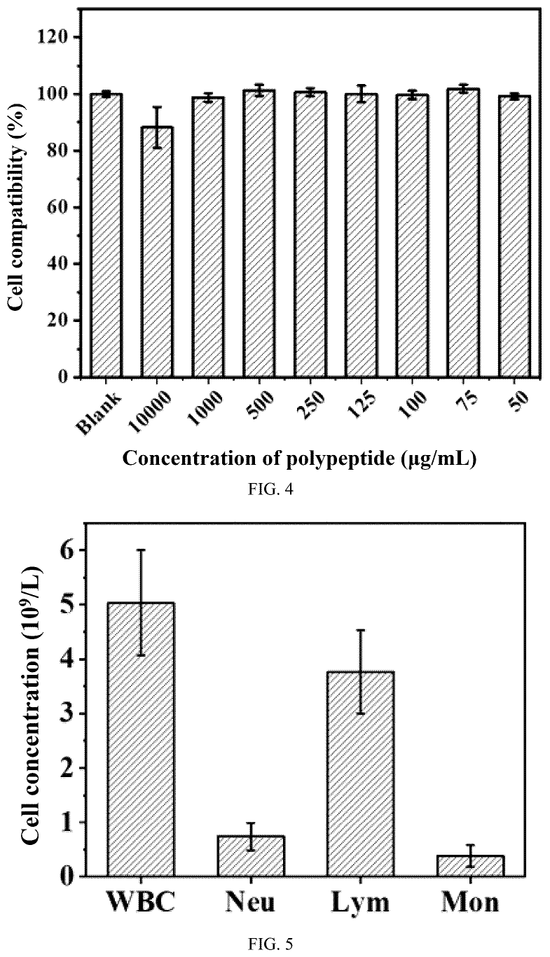

The polypeptide (prepared by Method 2) of the disclosure was added at different concentrations to Dulbecco's Modified Eagle's Medium (DMEM) containing fetal bovine serum (10% by volume) and used to culture fibroblasts. Cells were seeded at 10,000 cells per well in 96-well plates, incubated at 37° C. with a medium containing polypeptides at various concentrations as described above, and the cell viability was determined after 48 h using the Cell Counting Kit-8 method. The results are as shown in FIG. 4, which shows that the polypeptides disclosed herein are not cytotoxic in the effective concentration range.

### Example 4

A Nutritional Polypeptide Having Branched-Structure with Efficient Broad-Spectrum Antibacterial and Antifungal Functions has the Safety (Whether Acute Systemic Inflammation is Produced) Properties Demonstrated as Below:

The polypeptide (prepared by Method 1) of the present disclosure was dissolved in physiological saline at a concentration of 1 mg/mL and injected into rats through the caudal vein at a dose of 8 mg/kg. Experimental results showed that the rats were still alive after 24 h and no abnormal behavior was observed directly. Blood routine examination showed that the cell concentrations of white blood cells (WBC), neutrophils (Neu), monocytes (Mon) and lymphocytes (Lym) were all in the normal range (FIG. 5), exhibiting no inflammatory features. This result is better than most current cationic antibacterial agents and antibacterial polypeptides available in vivo for antibacterial treatment of systemic blood infections such as sepsis or septicemia.

### Example 5

A Nutritional Polypeptide Having Branched-Structure with Efficient Broad-Spectrum Antibacterial and Antifungal Functions has the In Vivo Hemocompatibility Properties Demonstrated as Follows:

The polypeptide (prepared by Method 1) of the present disclosure was dissolved in physiological saline at a concentration of 1 mg/mL and injected into rats through the caudal vein at a dose of 8 mg/kg. Experimental results showed that the rats survived 24 h and no abnormal behavior was observed directly. Blood routine examination showed that the concentrations of red blood cell (RBC), hemoglobin (HGB), and platelet (PLT) were all in the normal range (Table 1), exhibiting no abnormalities, demonstrating that it still has excellent hemocompatibility at high concentrations.

TABLE 1
Red blood cell concentration, hemoglobin content, and platelet
concentration in rat blood 24 h after injection of the branched-
structured polypeptide of the present disclosure at 1 mg/mL

| Blood | Red blood cell | Hemoglobin | Platelet |
|---|---|---|---|
| routine | concentration | content | concentration |
| index | (10¹²/L) | (g/L) | (10⁹/L) |
| Experimental | 6.3 ± 0.3 | 141.8 ± 10.1 | 847.0 ± 140.1 |
| group values | | | |
| Normal range | 6.36-9.42 | 110-143 | 450-1590 |

### Example 6

Method for Preparing an Antibacterial Non-Covalent Hydrogel Physically Embedded with Polypeptide:

The polypeptide (prepared by Method 1) according to the present disclosure and agarose were dissolved in 100 mL of deionized water at concentrations of 1 mg/mL and 30 mg/mL respectively at 90° C. After complete dissolution, the solution was poured into a circular culture dish and placed in a refrigerator at 4° C. to be cooled sufficiently to form an antimicrobial agarose gel containing the polypeptide. The non-covalent hydrogel physically embedded with the polypeptide has good antibacterial effects against Staphylococcus aureus, Escherichia coli, Pseudomonas aeruginosa, and Candida albicans.

### Example 7

Method for Preparing an Antibacterial Covalent Hydrogel Physically Embedded with Polypeptide:

The polypeptide (prepared by Method 2) according to the present disclosure, methacrylated gelatin and double-ended methacrylated polyethylene glycol were dissolved in 100 mL of deionized water at 90° C. at concentrations of 10 mg/mL and 10 mg/mL respectively. After complete dissolution, the solution was poured into a circular culture dish and placed in a refrigerator at 4° C. to be cooled sufficiently to form an antimicrobial covalent hydrogel containing the polypeptide. The covalent hydrogel physically embedded with the polypeptide has good antibacterial effects against Staphylococcus aureus, Escherichia coli, Pseudomonas aeruginosa, and Candida albicans.

### Example 8

Method for Preparing an Antimicrobial Polyurethane Electrospun Membrane Physically Embedded with Polypeptide:

The polypeptide (prepared by Method 1) according to the present disclosure and polyurethane were dissolved in hexafluoroisopropanol at 0.5% and 10% mass volume fractions respectively, where the polyurethane was synthesized from poly (ε-caprolactone) as a soft segment, hexamethylene diisocyanate as a hard segment, and ethylenediamine as a chain extender. The solution was added to a syringe and placed on a syringe pump of an electrospinning machine, the extrusion speed was set at 1 mL/h, the roller speed of the receiver was 300 rpm, and the positive and negative voltages were set at +15 kV and −8 kV, respectively. After 8 h of spinning, a polyurethane electrospun membrane physically embedded with the polypeptide was obtained. The polyurethane electrospun membrane showed good antibacterial effect after co-culture with Staphylococcus aureus after 24 h.

### Example 9

### Method for Preparing a Covalently Crosslinked Antibacterial Hydrogel:

Glycidyl methacrylate, hydroxyethyl methacrylate and benzoyl peroxide were dissolved in dimethyl sulfoxide at mass volume fractions of 1%, 5% and 0.1% respectively to give a hydrophilic polymer containing epoxy groups after thermally initiated polymerization at 50° C., purification by dialysis and lyophilization. The hydrophilic polymer and the polypeptide (prepared by Method 1) according to the present disclosure were dissolved in pure water at 10 mg/mL and 1 mg/mL respectively, and the solution system was left at 40° C. with stirring at normal temperature. After 24 h, the amino groups of the polypeptide and the epoxy of the polymer were cross-linked to form a hydrophilic polymer network, thereby constructing an antibacterial hydrogel.

### Example 10

Method for Preparing an Antibacterial Covalent Microgel Physically Entrapped with Polypeptides:

The polypeptide (prepared by Method 1), methacrylated gelatin and lithium phenyl-2,4,6-trimethylbenzoyl phosphite were dissolved in pure water at 1%, 10% and 0.05% respectively, dispersed in paraffin oil by ultrasound, polymerized by ultraviolet light to obtain microgel microparticles, which were then separated and purified to obtain an antibacterial covalent microgel.

Furthermore, the nutritional branched-structured polypeptides with highly efficient broad-spectrum antibacterial and antifungal functions based on the present disclosure can achieve innovative application effects in multiple related fields. For example, slow-release hydrogel ulcer patches can be produced, or antibacterial dressings that promote scar-free wound healing can be made. Specifically:

The slow-release hydrogel ulcer patch includes hydrogel, the polypeptides, analgesics, flavoring agents, coloring agents, and antioxidants. The concentration of the polypeptides is 10-200 mg/mL. The hydrogel is either an acrylic acid and acrylamide-based hydrogel or a polysaccharide hydrogel. The analgesic is at least one of ferulic acid, oleanolic acid, and ursolic acid. The flavoring agent is at least one of sucrose, mannitol, sorbitol, peppermint essential oil, and rose essential oil. The coloring agent is at least one of brilliant blue, tartrazine, carmine, and sunset yellow. The antioxidant is vitamin or natural plant extract, where the vitamin refers to vitamin C or vitamin E, and the natural plant extract refers to tea polyphenol or grape seed extract.

The preparation method of the ulcer patch is as follows:

Using acrylamide and acrylic acid as raw materials, ammonium persulfate/tetramethylethylenediamine as an initiator, and dimethylformamide as a cross-linking agent. Firstly, dissolving acrylamide and acrylic acid in a mass ratio of 2:10-2:20 in water under magnetic stirring to obtain a solution with a total concentration of 10-30 wt %, then adding dimethylformamide (MBA), ammonium persulfate (APS), and tetramethylethylenediamine (TEMED) successively and magnetic stirring to obtain a uniform solution, polymerizing to form a hydrogel after adding the polypeptides and auxiliary materials into the solution, spreading film to obtain a drug film, and cuting and sterilizing to obtain the hydrogel ulcer patch. This oral ulcer patch enhances the binding ability and durability between the polypeptides and the ulcer patch, inhibits the burst release of the polypeptides, thereby achieving a slow-release effect, greatly improving the drug efficacy. Moreover, using these nutritional branched-structured polypeptides for anti-inflammatory and sterilization purposes, it also has good biocompatibility and nutritional properties. The curative effect will not significantly decrease even after long-term use as a treatment material, having significant advantages over hormonal drugs and immunostimulants.

The antibacterial dressing that promotes scar-free wound healing includes the following components by weight: 0.05-0.1 parts of polypeptide anti-scar complex, 0.1-2.0 parts of carbomer, 1-10 parts of thickening agent, 2-12 parts of humectant, 150-200 parts of water, and 1-15 parts of plant extract. In addition, an alkali is adopted to adjust the pH to 6.8-7.2.

The preparation method of the polypeptide anti-scar complex is as follows:

The thiolated polypeptide and the anti-scar drug are synthesized into a complex through Michael addition. Specifically, preparing the polypeptide into an aqueous solution with a concentration of 10-30 mg/mL, mixing a fresh stock solution of 10-40 mg/mL 2-iminothiolane hydrochloride with the polypeptide solution at a volume ratio of 3:1 performing reaction at room temperature, rotary evaporating the reaction solution and repeatedly washing with isopropanol and rotary evaporating, and then drying in a vacuum oven until the mass is constant. Dissolving obtained thiol-modified hyperbranched polylysine and the anti-scar drug in water at a mass ratio of 1:1 to prepare a solution with a total concentration of 10-20 mg/mL, performing reaction at 50° C. for 5 hours, rotary evaporating the reaction solution and repeatedly washing with isopropanol and rotary evaporating, and then drying in a vacuum oven until the mass is constant, to obtain the polypeptide anti-scar complex.

The anti-scar drug is at least one of asiaticoside, salvianolic acid B, and papain. The thickening agent is at least one of polyvinyl alcohol, hypromellose, hydroxyethyl cellulose, and polyvinylpyrrolidone. The humectant is glycerol. The plant extract is at least one of aloe vera extract and cactus extract.

After mixing all the auxiliary materials, adding an alkali solution to adjust the pH to 6.8-7.2. Adding the polypeptide anti-scar complex to the obtained solution, magnetic stirring for uniform mixing, centrifugalizing and defoaming, to obtain the antibacterial dressing that promotes scar-free wound healing.

This scheme forms a composite drug by chemically grafting the branched-structured polypeptides with anti-scar drugs, making full use of the function that anti-scar drugs can target downstream proteins and other cell pathways related to scar formation, and carrying the polypeptides with antibacterial functions into relevant cell pathways. For example, salvianolic acid B targets the downstream effector protein CD36 to inhibit cell fibrosis at the wound site, enabling the composite drug to adhere to the cell surface, thereby achieving more targeted, deeper, and more persistent sterilization performance at the wound site. If the polypeptides and anti-scar drugs are simply mixed, the polypeptides will only act on the wound surface and cannot achieve the above effects. In addition, the overexpression of inflammatory factors is the key to scar formation. Utilizing the large number of terminal amino groups contained in the polypeptides to adsorb inflammatory factors containing acidic groups can directly play a role in slowing down or even eliminating the expression of inflammatory factors on the cell surface in the cell pathway, thereby further improving the scar-inhibiting function of the dressing.

### Example 11

A hydrogel was synthesized using acrylamide and acrylic acid as raw materials, APS/TEMED as an initiator, and MBA as a cross-linking agent. Firstly, acrylamide and acrylic acid were dissolved in a certain amount of water under magnetic stirring to obtain a solution with a concentration of 20 wt %. The mass ratio of acrylic acid to acrylamide was maintained at 2:13. Then, MBA, APS, and TEMED were added successively, and a uniform solution was obtained under magnetic stirring. The mass ratios of MBA, APS, and TEMED to the monomers were 0.1%, 0.9%, and 0.6 wt %, respectively. Subsequently, 20 mg/mL of the nutritional branched-structured polypeptides (prepared by Method 1), 5 mg/mL of analgesic, 5 mg/ml of flavoring agent, 5 mg/mL of coloring agent, and 5 mg/mL of antioxidant were added, stirred evenly, and after polymerization, the hydrogel was obtained. Then, a film was spread, dried to obtain a drug film, cut, and sterilized to obtain the hydrogel ulcer patch.

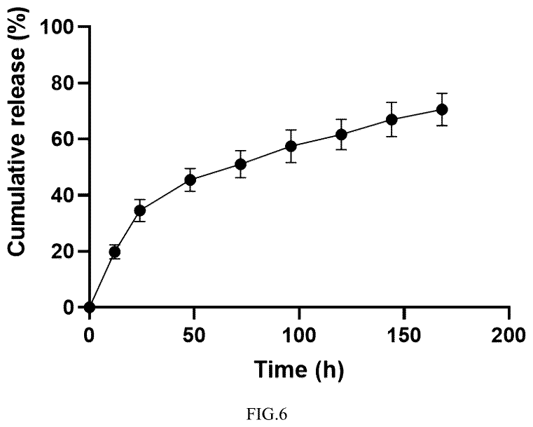

The release curve of the polypeptides within one week in this example is shown in FIG. 6. As shown in FIG. 6, due to the hydrogen bonding between the hydrogel and the polypeptides, the slow release of the polypeptides was achieved. Meanwhile, this ulcer patch has excellent antibacterial performance.

### Example 12

A certain amount of branched-structured polypeptides was taken and prepared into an aqueous solution with a concentration of 30 mg/mL. The fresh stock solution of 30 mg/mL 2-iminothiolane hydrochloride (2-IT) was mixed with the polypeptide solution at a volume ratio of 3:1, and the reaction was gently carried out at room temperature for two hours. The reaction solution was rotary evaporated and repeatedly washed with isopropanol by rotary evaporation, and then dried in a vacuum oven until the mass was constant (at 25° C.). Then, the obtained thiol-modified polypeptides and the anti-scar drug salvianolic acid B were dissolved in water at a mass ratio of 1:1 to prepare a solution with a concentration of 15 mg/mL. The reaction was carried out at 50° C. for 5 hours. The reaction solution was rotary evaporated and repeatedly washed with isopropanol by rotary evaporation, and then dried in a vacuum oven until the mass was constant (at 25° C.). Finally, the polypeptide anti-scar complex was obtained.

By weight, 3 parts of aloe vera extract, 7 parts of cactus extract, 15 parts of thickening agent, 0.1 part of the above-prepared polypeptide anti-scar complex, 10 parts of glycerol, and 80 parts of deionized water were weighed respectively, fully stirred and dissolved evenly for later use. 2.0 parts of carbomer 940 was added to 80 parts of water, heated to 60° C. to fully dissolve carbomer 940 in the water. After cooling to room temperature, it was poured into the above-prepared thickening agent solution, fully stirred and dissolved. Finally, an appropriate amount of pH regulator was added to adjust the pH to 6.8-7.2 to obtain an antibacterial gel dressing that promotes scar-free wound healing.

This gel dressing has good air permeability and moisture retention properties, as well as antibacterial, non-allergenic, safe, comfortable, natural, and environmentally friendly characteristics. This dressing also has the functions of antibacterial, anti-inflammatory, promoting wound healing, hemostasis, moisture absorption and retention, inhibiting scar hyperplasia, and good biocompatibility.

What is claimed is:

1. A nutritional polypeptide having branched-structure with efficient broad-spectrum antibacterial and antifungal functions, wherein the polypeptide is a polypeptide having a molecular weight of 3000-7000 Da and comprising L-lysine or D-lysine moieties, and has the following structural formula:

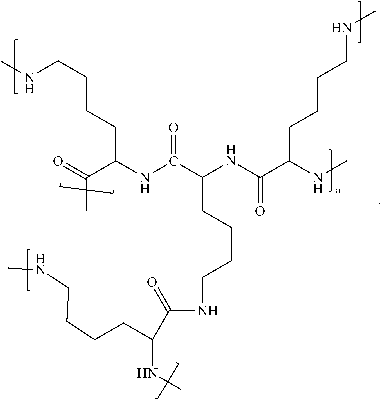

2. The nutritional polypeptide having branched-structure according to claim 1, wherein the polypeptide is capable of killing bacterial species comprising: (1) Gram-positive bacteria and drug-resistant variant strains thereof; and (2) Gram-negative bacteria and drug-resistant variant strains thereof.

3. The nutritional polypeptide having branched-structure according to claim 1, wherein the polypeptide is capable of killing fungal species comprising: (1) yeast fungi and drug-resistant variant strains thereof; and (4) mould fungi and drug-resistant variant strains thereof.

4. The nutritional polypeptide having branched-structure according to claim 1, wherein the polypeptide is capable of killing mycoplasma and drug-resistant variants thereof.

5. The nutritional polypeptide having branched-structure according to claim 1, wherein the polypeptide is used directly as a solid antibacterial agent.

6. The nutritional polypeptide having branched-structure according to claim 1, wherein the polypeptide is dissolved in an aqueous solution, an organic solvent or a mixed solvent thereof for use as a liquid antibacterial agent.

7. The nutritional polypeptide having branched-structure according to claim 1, wherein the polypeptide is prepared as an antibacterial complex by being carried on surface and/or inside of a carrier material by a physical and/or chemical action, the carrier material is a hydrogel, a metal or polymer substrate, a nanoparticle, or a microgel.

8. The nutritional polypeptide having branched-structure according to claim 4, wherein the polypeptide has a Minimum Inhibitory Concentration (MIC) of 10 μg/mL for an antibacterial effect of 90% or more.

9. The nutritional polypeptide having branched-structure according to claim 1, wherein the polypeptide is dissolved in physiological saline, and in a range of effective bactericidal concentration, intravenously or intramuscularly injected into an animal without adverse effect, which is used as an antibacterial agent for treatment of diseases related to blood infections.
